(19) United States
(12) Patent Application Publication
(10) Pub. No.: US 2020/0102438 A1
(43) Pub. Date: Apr. 2, 2020

(54) EXPANDABLE COMPOSITION COMPRISING A CYCLIC CARBONATE AND A BASE

(71) Applicant: Henkel AG & Co. KGaA, Duesseldorf (DE)

(72) Inventors: Sascha Poeller, Duesseldorf (DE); Rainer Kohlstrung, Plankstadt (DE); Thomas Rongier, Lyon 8 (FR)

(21) Appl. No.: 16/693,490

(22) Filed: Nov. 25, 2019

Related U.S. Application Data
(63) Continuation of application No. PCT/EP2018/066384, filed on Jun. 20, 2018.

(30) Foreign Application Priority Data
Jun. 21, 2017 (EP) 17177164.5

(51) Int. Cl. C08J 9/00; C07D 317/38; B29C 70/84

(52) U.S. Cl. CPC C08J 9/0033; C07D 317/38; C08J 9/0028; B29K 2063/00; C08J 2461/10; C08J 2363/00; B29C 70/845

(57) ABSTRACT

The present application relates to a thermally expandable composition which comprises at least one organic compound comprising at least two cyclic carbonate groups, at least one compound having at least one functional group with a pKa value of greater than 3 and at least one base, to shaped bodies which contain said composition, and to a method for sealing and filling voids in components, for strengthening or reinforcing components, in particular hollow components, and for bonding mobile components using shaped bodies of this type.

The present application relates to a thermally expandable composition which comprises at least one organic compound comprising at least two cyclic carbonate groups, at least one compound having at least one functional group having a pKa value of greater than 3 and at least one base, to shaped bodies containing this composition, and to a method for sealing and filling hollow spaces in components, for strengthening or stiffening components, in particular hollow components, and for bonding movable components, using shaped bodies of this kind.

Modern vehicles and vehicle parts have a plurality of hollow spaces which have to be sealed to prevent the entry of moisture and dirt, since this can lead to corrosion of the corresponding body parts from the inside out. This applies in particular to modern self-supporting body structures in which a heavy frame construction is replaced by lightweight, structurally stable frameworks made of prefabricated hollow space profiles. As a result of this system, constructions of this kind have a series of hollow spaces that have to be sealed against the ingress of moisture and dirt. Seals of this kind are also used for the purpose of preventing the transmission of airborne sound in hollow spaces of this kind, and thus to reduce unpleasant vehicle running noises and wind noises and thus to increase the driving comfort in the vehicle.

Baffle parts that cause a sealing and/or acoustic effect in hollow spaces of this kind are often referred to as “pillar fillers”, “baffles” or “acoustic baffles”. They usually consist either completely of thermally expandable shaped bodies or of shaped bodies containing a carrier and expandable polymeric compositions in the peripheral region thereof. These baffle parts are fastened to the open structures by means of hooking, clipping, screwing or welding during shell construction. After closing the structures during shell construction and further pretreating the body, the process heat of the furnaces for curing the cathodic dip coating is then exploited to trigger the expansion of the expandable part of the baffle part in order to thus seal the cross section of the hollow space.

Moreover, in modern vehicles, there is an increasing need for metal lightweight components that are intended for dimensionally consistent batch production and have predetermined rigidity and structural strength. In vehicle construction in particular, as a result of the desire to reduce weight, there is a need for metal lightweight components consisting of thin-walled sheets which still have adequate rigidity and structural strength. Shaped bodies made of thermally expandable compositions which impart the necessary support properties are also used in this case.

Corresponding thermally expandable compositions are described, for example, in the publications WO 2008/034755, WO 2007/039309, WO 2013/017536 and the German application 10 2012 221 192.6. These thermally expandable compositions are also used in the automotive sector.

Nowadays, blowing agents such as ADCA (azodicarbonamide), OBSH (4,4′-oxybis(benzenesulfonyl hydrazide)), DNPT (dinitroso pentamethylene tetramine), PTSS (p-toluene semicarbazide), BSH (benzene-4-sulfonohydrazide), TSH (toluene-4-sulfonohydrazide), 5-PT (5-phenyltetrazole) and the like are used in expandable compositions of this kind such as rubber vulcanizates (sulfur, peroxide or benzoquinone dioxime) for sealing and bonding ethylene vinyl acetate-based hollow space partitions, epoxy-based supporting foams and expandable sealants in automotive manufacturing.

These blowing agents are disadvantageous in that they can trigger respiratory sensitization, are generally questionable from a toxicological point of view, or are explosive. Furthermore, the decomposition of these blowing agents produces by-products such as ammonia, formamide, formaldehyde or nitrosamines, which are prohibited in automotive manufacturing in accordance with the Global Automotive Declarable Substance List (GADSL), the IFA (Institute for Occupational Health and Safety) CMR List August 2012 or the IFA Report “Index of hazardous substances 2012”. In addition, when using blowing agents, the VOC content (content of volatile organic compounds) is very high. These blowing agents have therefore been classified as SVHCs by the REACH Regulation and are likely to be banned. As an alternative to these blowing agents, physical blowing agents may be used which usually consist of a gas encapsulated by a polymer shell or slightly volatile substances such as pentane. However, the use of these blowing agents is disadvantageous because a relatively high mass of blowing agent has to be used to achieve high expansion rates. Furthermore, the expandable compositions must not contain any constituents which influence the stability of the polymer shell, because otherwise the blowing agent evaporates prior to its intended use, and this is also often highly flammable.

The problem addressed by the present invention was therefore that of providing thermally expandable compositions which are preferably storage-stable at room temperature and contain blowing agents which are neither explosive nor questionably toxic and which also have good expansion rates. In addition, the reactants should be integrated in the resulting foams as effectively as possible.

This problem is surprisingly solved by thermally expandable compositions which comprise
(1) at least one organic compound comprising at least two cyclic carbonate groups,
(2) at least one compound having at least one functional group having a pKa value of greater than 3, and
(3) at least one base.

Corresponding compositions overcome the known disadvantages while simultaneously exhibiting excellent expansion. In this case, the at least one organic compound comprising at least two cyclic carbonate groups acts both as a binder and as a blowing agent. Because the blowing agent simultaneously constitutes the binder, a very high and uniform expansion can be achieved. In addition, as a result, the blowing agent is ideally incorporated into the resulting binder backbone, since the blowing agent itself is the binder, and no unwanted volatile residues remain that could be toxic.

Therefore, in a first aspect, the present invention relates to a thermally expandable composition containing
(1) at least one organic compound comprising at least two cyclic carbonate groups;
(2) at least one compound having at least one functional group having a pKa value of greater than 3; and
(3) at least one base.

In a further aspect, the invention relates to a shaped body which comprises a thermally expandable composition according to the invention.

Furthermore, the present invention is directed to a method for sealing and filling hollow spaces in components, for strengthening or stiffening components, in particular hollow components, and for bonding movable components, using a thermally expandable composition or shaped body disclosed herein. In this case, the foaming of the expandable composition is caused by the at least one organic compound comprising at least one cyclic carbonate group, the expandable composition preferably expanding by more than 5 vol. %, preferably more than 50 vol. %, in particular more than 100 vol. %, in each case based on the expandable composition prior to expansion.

In yet a further aspect, the present invention relates to the use of a shaped body described herein for acoustically sealing hollow spaces in components and/or for sealing hollow spaces in components against water and/or moisture, or for strengthening or stiffening components, in particular hollow components.

“At least one”, as used herein, refers to one or more, for example 1, 2, 3, 4, 5, 6, 7, 8, 9 or more. In connection with constituents of the catalyst compositions described herein, this information does not refer to the absolute amount of molecules, but to the type of the constituent. “At least one organic carbonate” therefore means, for example, one or more organic carbonates, i.e. one or more different types of organic carbonates. Together with stated quantities, the stated amounts refer to the total amount of the correspondingly designated type of constituent, as defined above.

“Integer”, as used herein, is 0 and all natural numbers. “Natural number”, as used herein, refers to the general meaning of the term and is 1, 2, 3, 4, 5, 6, 7, 8, 9, 10 and more.

As an essential constituent, the compositions contain at least one organic compound comprising at least two cyclic carbonate groups, which is advantageous in that it is neither harmful to health nor explosive and that no volatile organic compounds (VOCs) are produced during expansion. The decomposition products are substantially CO₂ and the organic “remainder of the molecule” to which the cyclic carbonate group was bonded. The organic “remainder of the molecule” then forms the binder system such that volatile organic compounds are not released. Furthermore, the products produced therewith have a uniform foam structure over the entire process temperature range used for curing.

In this case, the at least one organic compound comprising at least two cyclic carbonate groups acts both as a binder and as a blowing agent. A chemical blowing agent is understood, according to the invention, to mean compounds which decompose upon exposure to heat and thereby release gases. The at least one organic compound comprises at least two cyclic carbonate groups bonded to the organic compound. The cyclic carbonate groups are necessarily bonded to an organic compound. A compound consisting solely of a cyclic carbonate group, such as ethylene carbonate, is not a compound according to the invention. During decomposition, purely cyclic carbonates as blowing agents, such as ethylene carbonate, leave behind volatile organic compounds which cannot be integrated or can only be poorly integrated in the foamed composition and also cannot be used as a binder.

The organic compound preferably comprises at least two to four cyclic carbonate groups. The organic compound is preferably a polyether and/or polyester, in particular a polyether, to which at least one, preferably at least two cyclic carbonate groups are bonded.

In a further preferred embodiment, the organic compound comprising at least two cyclic carbonate groups is a compound of Formula (I)

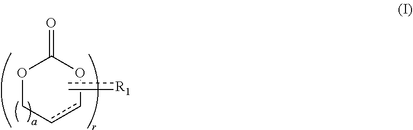

In this case [symbol] represents a single or double bond, preferably a single bond. It is clear that, if the ring contains a double bond, R₁ is not bonded by an exo double bond but by a single bond, and vice versa. R₁ is a linear or branched, substituted or unsubstituted alkyl, linear or branched, substituted or unsubstituted heteroalkyl, linear or branched, substituted or unsubstituted alkenyl, linear or branched, substituted or unsubstituted alkinyl, substituted or unsubstituted cycloalkyl, substituted or unsubstituted aryl, or —C(O)—R^{a}, where R^{a} is H, a linear or branched, substituted or unsubstituted alkyl, linear or branched, substituted or unsubstituted heteroalkyl, linear or branched, substituted or unsubstituted alkenyl, linear or branched, substituted or unsubstituted alkinyl, substituted or unsubstituted cycloalkyl, substituted or unsubstituted aryl, preferably a linear or branched, substituted or unsubstituted heteroalkyl, R₁ preferably comprising at least 2, preferably at least 4 carbon atoms. In Formula (I), a is an integer from 0 to 5, preferably 0 or 1 and particularly preferably 0. Moreover, r is a natural number from 2 to 10, preferably from 2 to 4.

“Substituted”, as used herein in connection with the alkyl or heteroalkyl groups, means that the corresponding group having one or more substituents selected from the group consisting of —OR′, —COOR′, —NRR″, —C(═X)NR′R″, —NR″C(═X)NR′R″, halogen, unsubstituted C₆₋₁₄ aryl, unsubstituted C₂₋₁₄ heteroaryl containing 1 to 5 heteroatoms selected from O, N and S, unsubstituted C₃₋₁₀ cycloalkyl and unsubstituted C₂₋₁₀ heteroalicyclyl containing 1 to 5 heteroatoms selected from O, N and S is substituted.

“Substituted”, as used herein in connection with the aryl and cycloalkyl groups, means that the corresponding group having one or more substituents selected from the group consisting of —OR′, —COOR′, —NR′R″, —C(═X)NR′R″, —NR″C(═X)NR′R″, halogen, unsubstituted C₁₋₁₀ alkyl and —CH₂ aryl may be substituted, it being possible to in turn substitute the aryl group in the —CH₂ aryl group with —OR′, —COOR′, —NR′R″, —C(═X)NR′R″, —NR″C(═X)NR′R″, halogen and unsubstituted C₁₋₁₀ alkyl.

R′ and R″ are in this case selected, independently of one another, from H, unsubstituted C₁₋₁₀ alkyl, unsubstituted C₆₋₁₄ aryl, unsubstituted C₂₋₁₄ heteroaryl, unsubstituted C₃₋₁₀ cycloalkyl, unsubstituted C₂₋₁₀ heteroalicyclyl, alkylaryl, arylalkyl, heteroarylalkyl and alkylheteroaryl.

“Alkyl”, as used herein, refers to linear or branched alkyl groups preferably having 1 to 30 carbon atoms, such as methyl, ethyl, n-propyl, iso-propyl, n-butyl, n-pentyl, n-hexyl, n-heptyl, n-octyl, n-nonyl, n-decyl, n-undecyl and n-dodecyl and the linear C₁₄, C₁₆ and C₁₈ alkyl groups. In various embodiments, the alkyl groups are short-chain C₁₋₄ alkyl groups, in particular unsubstituted, linear C₁₋₄ alkyl groups. The alkyl groups may be substituted or unsubstituted, but are preferably unsubstituted. When substituted, the substituents are selected in particular from the group of substituents described above.

“Heteroalkyl”, as used herein, refers to alkyl groups as defined above in which at least one carbon atom is replaced by a heteroatom, in particular N or O, particularly preferably O.

“Aryl”, as used herein, refers to aromatic groups having 5 to 30 carbon atoms which may have at least one aromatic ring, but also a plurality of condensed rings, such as phenyl, naphthyl, anthracenyl, and the like. The aryl groups may be substituted or unsubstituted. When substituted, the substituents are selected from the group described above.

“Heteroaryl”, as used herein, refers to aryl groups as defined above in which at least one carbon atom is replaced by a heteroatom, in particular N, S or O, particularly preferably O.

“Halogen”, as used herein, refers to F, Cl, Br and I.

“Cycloalkyl”, as used herein, refers to non-aromatic, cyclic hydrocarbons, in particular cyclic alkyl or alkenyl groups as defined above, e.g. cyclopentyl, cyclohexyl and cyclohexenyl groups. When substituted, the substituents are selected from the group described above.

“Heteroalicyclyl”, as used herein, refers to cycloalkyl groups as defined above in which at least one carbon atom is replaced by a heteroatom, in particular N, S or O, particularly preferably O.

In another preferred embodiment, the organic compound comprising at least two cyclic carbonate groups is a compound of Formula (II)

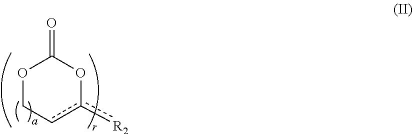

In Formula (II), [symbol] , a and r are as defined above. Furthermore, R₂ is defined as R₁. In this case it is also clear that, if the ring contains a double bond, R₂ is not bonded by an exo double bond but by a single bond, and vice versa. R₂ is also defined as R₁ and preferably a linear or branched, substituted or unsubstituted heteroalkyl.

Compounds in which R₁ or R₂ is a divalent or polyvalent heteroalkyl group, for example a pentaerythritol group, to which 2 or more of the cyclic carbonate groups are then bonded, are particularly preferred. In particular, R₁ and R₂ represent polyesters and polyethers, preferably polyethers, to which 2 or more of the cyclic carbonate groups are then bonded.

Cyclic carbonates which correspond to derivatives of the carbonates of the Formulae (I) and (II) are frequently used to prepare alkenyl ether polyols. Examples of derivatives thereof include those which are substituted on the ring methylene groups, in particular those that do not carry the R₁ or R₂ group, for example by organic groups, in particular linear or branched, substituted or unsubstituted alkyl or alkenyl groups having up to 20 carbon atoms, in particular ═CH₂ and —CH═CH₂, or linear or branched, substituted or unsubstituted heteroalkyl or heteroalkenyl groups having up to 20 carbon atoms and at least one oxygen or nitrogen atom, or functional groups such as —OH or —COOH. Examples of derivatives of this kind include, for example, 4-methylene-1,3-dioxolan-2-one, which carries the R₁ or R₂ group at the fifth position, or di-(trimethylolpropane) dicarbonate, the R₁ or R₂ group in the fifth position being a methylene-trimethylol monocarbonate group.

In various embodiments in which the R₁ or R₂ group is bonded by means of a single bond, the ring carbon atom carrying the R₁ or R₂ group can be substituted by another substituent, which is defined in the same way as the above-mentioned substituents for the other ring methylene group.

Other derivatives are those in which one or both of the ring oxygen atoms are replaced by sulfur atoms, and those in which, alternatively or in addition, the carbonyl oxygen atom is replaced by a sulfur atom.

The organic compounds comprising at least two cyclic carbonate groups are preferably prepared by reacting an epoxide or an oxetane with carbon dioxide; in particular the compounds of Formula (II) are prepared in this way. When reacting an epoxide, a compound comprising a five-membered cyclic carbonate is obtained, whereas when reacting an oxetane, a compound comprising a six-membered cyclic carbonate is obtained. Particularly preferred are the used organic compounds comprising at least two cyclic carbonate groups prepared by reacting an epoxide, preferably a polyepoxide, in particular the polyepoxides mentioned later, with carbon dioxide. Epoxides based on a polyhydric aliphatic polyether, such as ethers of pentaerythritol, or aromatic epoxides, in particular epoxides based on bisphenol derivatives, are particularly preferred. When reacting a polyepoxide (compound having at least two, in particular precisely two epoxide groups per molecule) with carbon dioxide, the reaction does not necessarily have to be complete; 70 to 100% of the epoxide groups is preferably reacted. However, a full reaction is preferred.

In an even more preferred embodiment of the present invention, the organic compound comprising at least two cyclic carbonate groups is a compound of Formula (III) or (IV).

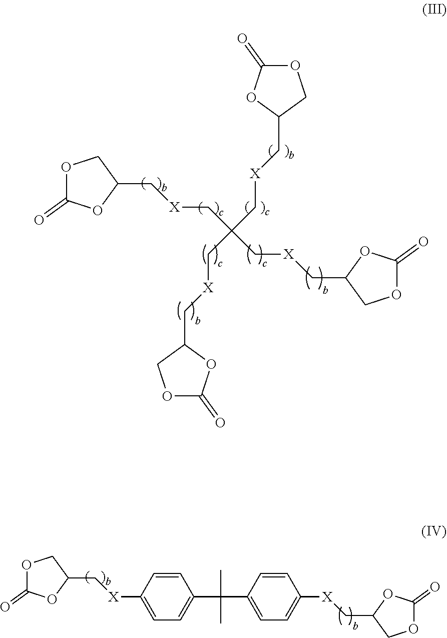

In this case, each b and each c are, independently of one another, a natural number from 1 to 5, preferably 1 or 2 and particularly preferably 1. Each b can be the same as or different to each c. Each b and each c is preferably the same. Furthermore, each X is independently selected from the group consisting of O and S. Preferably, each X is O.

In an even more preferred embodiment, the organic compound comprising at least one cyclic carbonate group is a compound of Formula (V) or (VI).

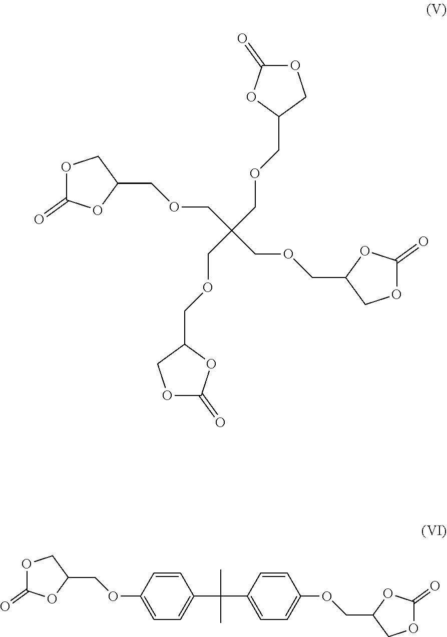 [SMILES: O=C1OCC(COCC(COCC2COC(=O)O2)(COCC2COC(=O)O2)COCC2COC(=O)O2)O1 ; CC(C)(c1ccc(OCC2COC(=O)O2)cc1)c1ccc(OCC2COC(=O)O2)cc1]

The organic carbonates according to the invention are synthetically accessible via a corresponding epoxide and CO₂ as starting materials in the presence of a catalyst under mild conditions.

The thermally expandable compositions preferably contain the organic compounds comprising at least two cyclic carbonate groups in an amount of at least 20 wt. %, in particular at least 25 wt. %, or preferably in an amount of from 20 to 90 wt. %, preferably 25 to 80 wt. %, particularly preferably 25 to 75 wt. %, even more preferably 25 to 70 wt. %, and most preferably 30 to 65 wt. %. Unless indicated otherwise, the amounts in wt. % given here are based on the total weight of the total composition prior to expansion.

In another preferred embodiment, the thermally expandable compositions preferably contain the organic compounds which are used as blowing agents and binders and comprise at least two cyclic carbonate groups in an amount of at least 20 wt. %, in particular at least 25 wt. %, or preferably in an amount of 20 to 90 wt. %, preferably 25 to 80 wt. %, particularly preferably 25 to 75 wt. %, even more preferably 25 to 70 wt. % and most preferably 30 to 65 wt. %, in each case based on the binder system (total weight of the total composition prior to expansion without fillers).

Optionally, further chemical blowing agents may be added to the composition according to the invention.

The expandable compositions are preferably free of ADCA (azodicarbonamide) and OBSH (4,4′-oxybis(benzenesulfonyl hydrazide)), in particular free of ADCA (azodicarbonamide), OBSH (4,4′-oxybis(benzenesulfonyl hydrazide)), DNPT (dinitroso pentamethylene tetramine), PTSS (p-toluene semicarbazide), BSH (benzene-4-sulfonohydrazide), TSH (toluene-4-sulfonohydrazide) and 5-PT (5-phenyltetrazole), particularly preferably free of other exothermic blowing agents, in particular free of other blowing agents. The expandable compositions are preferably free of inorganic carbonates. “Free of”, as used in this context, means that the amount of the corresponding substance in the reaction mixture is less than 0.05 wt. %, preferably less than 0.01 wt. %, more preferably less than 0.001 wt. %, based on the total weight of the reaction mixture, in particular completely free.

In general, the expandable compositions may contain further binders in addition to the described at least one organic compound comprising at least two cyclic carbonate groups. In a preferred embodiment, the composition also contains at least one further reactive binder, in particular in combination with at least one curing agent and/or accelerator. The further reactive binder is preferably selected from the group of epoxides, rubbers and peroxidically crosslinkable polymers, in particular epoxides.

A preferred subject contains epoxides as another reactive binder. A large number of polyepoxides having at least 2 1,2-epoxy groups per molecule are suitable as epoxy resins. The epoxide equivalent of these polyepoxides can vary between 150 and 50,000, preferably between 170 and 5,000. In principle, the polyepoxides may be saturated, unsaturated, cyclic or acyclic, aliphatic, alicyclic, aromatic or heterocyclic polyepoxide compounds. Examples of suitable polyepoxides include polyglycidyl ethers prepared by reacting epichlorohydrin or epibromohydrin with a polyphenol in the presence of an alkali. Polyphenols suitable for this are, for example, resorcinol, pyrocatechol, hydroquinone, bisphenol A (bis-(4-hydroxy-phenyl)-2,2-propane), bisphenol F (bis(4-hydroxyphenyl)methane), bis(4-hydroxyphenyl)-1,1-isobutane, 4,4′-dihydroxybenzophenone, bis(4-hydroxyphenyl)-1,1-ethane and 1,5-hydroxynaphthaline. Other polyphenols that are suitable as the basis for polyglycidyl ethers are the known condensation products of phenol and formaldehyde or acetaldehyde of the novolac resin type.

Other polyepoxides that are in principle suitable are the polyglycidyl ethers of polyalcohols or diamines. These polyglycidyl ethers are derived from polyalcohols such as ethylene glycol, diethylene glycol, triethylene glycol, 1,2-propylene glycol, 1,4-butylene glycol, triethylene glycol, 1,5-pentanediol, 1,6-hexanediol or trimethylolpropane.

Other polyepoxides are polyglycidyl esters of polycarboxylic acids, for example reaction products of glycidol or epichlorohydrin with aliphatic or aromatic polycarboxylic acids such as oxalic acid, succinic acid, glutaric acid, terephthalic acid or dimer fatty acid.

Other epoxides are derived from the epoxidation products of olefinically unsaturated cycloaliphatic compounds or from native oils and fats.

Depending on the intended use, it can be preferable for the composition to additionally contain a flexibilizing resin. This may also be an epoxy resin. The inherently known adducts of carboxyl-terminated butadiene-acrylonitrile copolymers (CTBN) and liquid epoxy resins based on the diglycidyl ether of bisphenol A can be used as flexibilizing epoxy resins. Specific examples are the reaction products of Hycar CTBN 1300×8, 1300×13 or 1300×15 from BF Goodrich with liquid epoxy resins. Furthermore, the reaction products of amino-terminated polyalkylene glycols (Jeffamine) can also be used with an excess of liquid polyepoxides. In principle, according to the invention, reaction products of mercapto-functional prepolymers or liquid thiol polymers can also be used with an excess of polyepoxides as flexibilizing epoxy resins. However, the reaction products of polymeric fatty acids, in particular dimer fatty acid, with epichlorohydrin, glycidol or in particular diglycidyl ether of bisphenol A (DGBA) are very particularly preferred.

The thermally expandable preparations preferably contain at least 2 wt. % of at least one further epoxide. Thermally expandable preparations containing from 2 to 50 wt. %, in particular from 5 to 30 wt. %, of at least one epoxide, in each case based on the total mass of the thermally expandable preparation, are particularly preferred.

Guanidines, substituted guanidines, melamine resins, guanamine derivatives, cyclic tertiary amines, aromatic amines and/or mixtures thereof can be used as thermally activatable or latent curing agents for the epoxy resin binder system consisting of the aforementioned components. In this case, the curing agents can be stoichiometrically involved in the curing reaction. However, they may also have a catalytic effect. Examples of substituted guanidines are methylguanidine, dimethylguanidine, trimethylguanidine, tetramethylguanidine, methylisobiguanidine, dimethylisobiguanidine, tetramethylisobiguanidine, hexamethylisobiguanidine, heptamethylisobiguanidine, and very particularly cyanoguanidine (dicyandiamide). Representatives of suitable guanamine derivatives which may be mentioned are alkylated benzoguanamine resins, benzoguanamine resins or methoxymethyl-ethoxymethylbenzoguanamine. For monocomponent, heat-curing shaped bodies, the selection criterion is the low solubility of these substances at room temperature in the resin system, and therefore solid, finely ground curing agents are preferred in this case. Dicyandiamide is particularly suitable. Good storage stability of the heat-curing shaped bodies is thereby ensured.

Preferably, the curing agent and/or accelerator is present in a total amount of at least 0.25 wt. %, and in particular at least 1.5 wt. %, based on the total composition prior to expansion. However, a total of more than 5 wt. %, based on the total mass of the composition, is generally not required.

In order to improve shock resistance, one or more “impact modifiers” may also be present, which are known in the prior art for this purpose. Examples are thermoplastic resins which preferably carry groups that are reactive to epoxy groups. Natural or synthetic rubbers are also suitable for this purpose. Specific examples of these can be found in paragraphs [27] and [28] (pages 6 and 7) of WO 2007/004184.

As a further constituent, the thermally expandable composition comprises at least one compound having at least one functional group, in particular at least two functional groups, having a pKa value of greater than 3, preferably greater than 5, in particular greater than 7, preferably greater than 9. For example, this compound could be a novolac (phenolic resin) having aromatic OH groups as functional groups, pure phenol having a pKa value of approximately 10. The at least one functional group preferably has a pKa value of less than 30, in particular less than 25, preferably less than 20.

The compound having at least one functional group is typically deprotonated by the base and acts as a weak nucleophile, which allows the ring opening of the carbonates and thus the release of CO₂ In addition, the functional groups can react with the carbonate and thus build up the binder system. However, excessively strong nucleophiles do not generally cause carbon dioxide to be released. This compound should preferably not have a functional group having a pKa value of less than 3 or a hydrogen having a pKa value of less than 3.

The expandable composition preferably contains a compound of at least one, preferably at least two functional groups selected from OH, NH₂ and/or SH groups. Compounds having at least one, preferably at least two, functional groups selected from aromatic OH, aromatic NH₂ and/or aliphatic SH groups are particularly preferred. The compositions particularly preferably contain at least one polyol, polythiol, polyamine or mixtures thereof, in particular aromatic polyols, aromatic polyamines and/or aliphatic polythiols. “Aromatic” in this case means that, for example, the OH group is bonded directly to an aromatic system such that the negative charge can be stabilized by mesomeric rearrangement after deprotonation of the OH group.

Preferred alcohols are compounds which have at least one OH group per molecule, preferably at least two OH groups (polyol). An OH group is a free OH group. Preferred polyols are aromatic polyols, in particular polyphenols or novolac resins. Novolac resins are condensates of phenols with formaldehyde. Phenol novolac and/or cresol novolac are particularly preferred as polyols.

Preferred alcohols, in particular polyols, have a molecular weight of greater than 100 g/mol, preferably from 100 to 20,000 g/mol, more preferably from 200 to 10,000 g/mol, even more preferably from 400 to 5,000 g/mol. Novolac resins, in particular phenol novolac and/or cresol novolac, having a molecular weight of from 200 to 20,000 g/mol, preferably 300 to 10,000 g/mol, more preferably 400 to 5,000 g/mol, are very particularly preferred. Where reference is made in this document to molecular weights of polymeric compounds, the figures refer to the number-average molecular weight Mₙ, unless specified otherwise. The molecular weight can be determined by means of GPC against a polystyrene standard.

Preferred amines are compounds which have at least one NH₂ group per molecule, preferably at least two NH₂ groups (polyamine). An amine group is a free NH₂ group. Preferred amines are aromatic amines, in particular aromatic polyamines. Aniline is preferred as the amine.

Preferred thiols are compounds which have at least one thiol group per molecule, preferably at least two thiol groups (polythiol). A thiol group is a free SH group. Preferred polythiols have 2 to 10 thiol groups, in particular 2 to 6, very particularly preferably 2 to 4, above all 3 or 4 thiol groups. Thiols which themselves are a polyether or polyester are preferably used. Particularly preferred thiols are simple polyesters of a preferably tri- or tetrafunctional alcohol with 3-thiolpropanoic acid.

Examples of polythiols are listed in the following: 1,2-ethanedithiol, 1,3-propanedithiol, 1,2-propanedithiol, 1,4-butanedithiol, 1,3-butanedithiol, 2,3-butanedithiol, 1,5-pentanedithiol, 1,3-pentanedithiol, 1,6-hexanedithiol, 1,3-dithio-3-methylbutane, ethylcyclohexyldithiol, ethylcyclohexyldithiol, methylcyclohexyldithiol, methyl-substituted dimercaptodiethylsulfide, dimethyl-substituted dimercaptodiethylsulfide, 2,3-dimercapto-1-propanol, bis-(4-mercaptomethylphenyl) ether, 2,2′-thiodiethanethiol, pentaerythritol tetrakis (3-mercaptobutylate), dipentendimercaptan, trimethylopropane tris-3-mercaptopropionate, pentaerythritol tetra-3-mercaptopropionate and ethoxylated trimethylpropane tris-3-mercaptopropionate. Trimethylopropane tris-3-mercaptopropionate, pentaerythritol tetra-3-mercaptopropionate and ethoxylated trimethylpropane tris-3-mercaptopropionate are particularly preferred.

Compounds having at least two functional groups selected from OH and/or SH groups, in particular having a pKa value of greater than 5, in particular from 5 to 20, preferably from 8 to 14, are particularly preferred.

The thermally expandable compositions preferably contain this at least one compound having at least one functional group in an amount of 10 to 80 wt. %, preferably 15 to 70 wt. %, particularly preferably 20 to 60 wt. %, even more preferably 25% to 50 wt. %. Unless indicated otherwise, the amounts in wt. % given here are based on the total weight of the total composition prior to expansion.

In another preferred embodiment, the thermally expandable compositions preferably contain this at least one compound having at least one functional group in an amount of 10 to 80 wt. %, preferably 15 to 70 wt. %, particularly preferably 20 to 60 wt. %, even more preferably 25% to 50 wt. %, in each case based on the binder system (total weight of the total composition prior to expansion without fillers).

The thermally expandable composition comprises at least one base as a further constituent. The base allows foaming of the expandable composition. The adopted mechanism is in this case that the base deprotonates/activates the compound having a functional group and causes it to release CO₂ by nucleophilic attack of the carbonate. The base should thus preferably be suitable for deprotonating or activating the compound having at least one functional group, in particular when the temperature is increased.

Both inorganic and organic bases can be used as the base. In a preferred embodiment, the composition contains at least one inorganic base. Suitable inorganic bases are, for example, hydrides or hydroxides, in particular of alkali metals and/or alkaline-earth metals. Sodium or lithium hydrides or hydroxides, in particular sodium hydride or sodium hydroxide, are particularly preferred.

In another preferred embodiment, the composition contains at least one organic base. In this case it is preferred for at least one nitrogen-containing base to be contained. The nitrogen-containing base may be an ionic or non-ionic compound.

Preferred ionic bases are derivatives of imidazolium compounds, in particular 1,3-substituted imidazolium compounds. Preferred compounds are 1-ethyl-3-methyl-1H-imidazolium acetate, 1-ethyl-3-methyl-1H-imidazolium thiocyanate, 1-ethyl-3-methyl-1H-imidazolium cyanocyanamide, 1-ethyl-3-methyl-1H-imidazolium diethyl phosphate and 1,3-bis(2,6-diisopropylphenyl)-1H-imidazolidinium chloride.

Preferred non-ionic bases are non-ionic, nitrogen-containing bases which contain at least one tertiary nitrogen atom and/or an imine nitrogen atom.

The term “tertiary”, as used herein, indicates that three organic functional groups are covalently bonded, by means of single bonds, to the nitrogen atom that is contained in the at least one base.

Alternatively, the at least one base may contain an imine nitrogen atom. The term “imine”, as used herein, refers to the known substance class and indicates that the nitrogen atom has a covalent double bond to an organic group and a covalent single bond to another organic group. Imines are Schiff's bases.

The adhesive composition can contain a plurality of the above-described non-ionic bases, for example a base comprising an imine nitrogen and a base comprising a tertiary nitrogen atom. The non-ionic base can also be both a tertiary amine and an imine by containing both a tertiary nitrogen atom and an imine nitrogen.

In preferred embodiments, the at least one non-ionic base is a tertiary amine of Formula (BI):

NR₃R₄R₅ (BI)

and/or an imine of Formula (BII):

N(═R₆)R₇ (BII)

wherein the R₃ to R₅ and R₇ groups are each selected, independently of one another, from the group consisting of substituted or unsubstituted, linear or branched alkyl having 1 to 20 carbon atoms, substituted or unsubstituted, linear or branched alkenyl having 3 to 20 carbon atoms and substituted or unsubstituted aryl having 5 to 20 carbon atoms, or
wherein at least two of R₃ to R₅ form, together with the nitrogen atom to which they are bonded, a 5- to 10-membered heteroalicyclic ring or heteroaryl ring, which optionally contains one or more additional nitrogen atoms, in particular 1 additional nitrogen atom.
wherein R₆ is a substituted or unsubstituted, linear or branched alkylenyl having 3 to 20 carbon atoms, or R₃ and R₄ form, together with the nitrogen atom to which they are bonded, a 5- to 10-membered heteroalicyclic ring or heteroaryl ring, which optionally contains additional nitrogen atoms.

“Alkylenyl”, as used herein, refers to an alkyl group which is bonded to the nitrogen atom by a double bond. If substituted, the substituents are defined as described above for alkyl groups.

In various embodiments of the invention, the tertiary amine bases or the imine bases are cyclic compounds which contain at least two nitrogen atoms, i.e. at least two of the R₃ to R₇ groups combine in order to form, together with the nitrogen atom to which they are bonded, a ring, and further contain an additional nitrogen atom in the form of an —NRR′ group, the nitrogen atom being a ring atom and the R or R′ group being part of the ring formation. Bases based on imidazole or imidazolidine are particularly preferred. In various embodiments, the bases are therefore imidazole derivatives such as 1-alkyl-imidazole or 2,4-dialkylimidazole, for example.

In various embodiments, the at least one non-ionic base is selected from the group consisting of 1-methylimidazole, 2,4-ethylmethylimidazole, 1,4-diazabicyclo[2.2.2]octane (DABCO), 1,8-diazabicyclo[5.4.0]undec-7-ene (DBU) and the mixtures thereof.

Preferred bases are shown in the following.

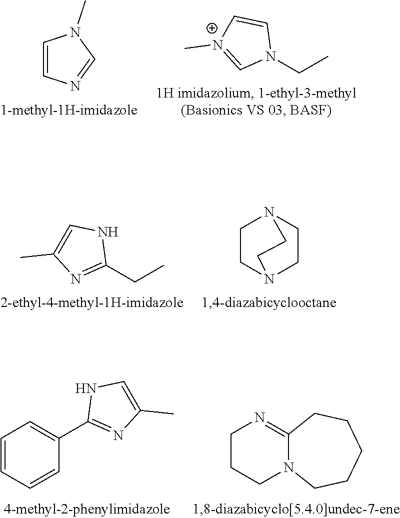

The base is particularly preferably selected from the group consisting of 1-methylimidazole, 2,4-ethylmethylimidazole, 1-ethyl-3-methylimidazole and 4-methyl-2-phenylimidazole.

In particularly preferred embodiments, the base is a non-ionic nitrogen-containing base, in particular selected from the group consisting of 1-methylimidazole, 2,4-ethylmethylimidazole (EMI), 1,4-diazabicyclo[2.2.2]octane (DABCO), 1,8-diazabicyclo[5.4.0]undec-7-ene (DBU), 1,5-diazobicyclo[3.4.0]non-5-ene (DBN) and mixtures thereof. The base is preferably selected from the group consisting of EMI, DBU and mixtures thereof.

In a further preferred embodiment, a urea or a urea derivative is contained as the base. The composition particularly preferably contains a substituted urea. “Substituted” in this context preferably means that the urea on the nitrogen atoms has one or more substituents selected from the group consisting of a substituted or unsubstituted, linear or branched alkyl having 1 to 20 carbon atoms, substituted or unsubstituted, linear or branched alkenyl having 3 to 20 carbon atoms and substituted or unsubstituted aryl having 5 to 20 carbon atoms. Preferred examples are 1,1-dimethylurea, p-chlorophenyl-N,N-dimethylurea (monuron), 3-phenyl-1,1-dimethylurea (fenuron) or 3,4-dichlorophenyl-N,N-dimethylurea (diuron). A urea, in particular a substituted urea, is particularly preferred, since it is assumed that the urea can additionally open the carbonates by a separate mechanism, it also being possible for polyurethane groups to be produced which can advantageously act on the binder system.

In a preferred embodiment, the compositions contain, in each case based on the binder system, in particular in each case based on the total weight, 0.01 to 10 wt. %, preferably 0.1 to 5 wt. %, more preferably 0.1 to 2 wt. %, of the at least one base. In another preferred embodiment, the compositions contain, based on the total weight, 0.01 to 10 wt. %, preferably 0.5 to 7 wt. %, more preferably 1 to 5 wt. %, of at least one urea or a urea derivative.

In a preferred embodiment, the thermally expandable composition contains
(1) at least one organic compound comprising at least two cyclic carbonate groups, in particular at least one organic compound of Formula (III) or (IV);
(2) at least one compound having at least one functional group, in particular having at least two functional groups selected from the group of OH, NH₂ and/or SH groups, in particular at least one compound selected from polyols, polythiols and/or polyamines; and
(3) at least one urea and/or a urea derivative.

In another preferred embodiment, the thermally expandable composition contains
(1) at least one organic compound comprising at least two cyclic carbonate groups, in particular at least one organic compound of Formula (III) or (IV);
(2) optionally at least one further epoxide
(3) at least one compound having at least two functional groups selected from OH and/or SH groups, in particular having a pKa value of greater than 3, preferably greater than 5, in particular from 5 to 20, preferably from 8 to 14; and
(4) at least one base.

In a further preferred embodiment, the thermally expandable composition contains, in each case based on the binder system, in particular in each case based on the total weight of the composition prior to expansion:
(1) at least 20 wt. %, in particular at least 25 wt. %, or preferably 20 to 90 wt. %, in particular 25 to 80 wt. %, particularly preferably 25 to 75 wt. %, even more preferably 25 to 70 wt. % and most preferably 30 to 65 wt. %, of at least one organic compound comprising at least two cyclic carbonate groups, in particular at least one organic compound of Formula (III) or (IV);
(2) 10 to 80 wt. %, preferably 15 to 70 wt. %, particularly preferably 20 to 60 wt. %, even more preferably 25 to 50 wt. %, of at least one compound having at least one functional group having a pKa value of greater than 3, preferably at least one polyol, polythiol, polyamine or mixtures thereof; and
(3) 0.01 to 10 wt. %, preferably 0.1 to 5 wt. %, more preferably 0.1 to 2 wt. %, of at least one base, preferably a nitrogen-containing base.

In addition to the above-mentioned constituents, the thermally expandable compounds may contain further conventional components, such as fillers, plasticizers, reactive diluents, rheology auxiliary agents, wetting agents, adhesion promoters, anti-ageing agents, stabilizers, and/or dye pigments.

Fillers include, for example, the various ground or precipitated chalks, calcium magnesium carbonates, talc, graphite, barite, silicic acid or silica and in particular silicate fillers such as mica, for example in the form of chlorite, or silicate fillers of the aluminum-magnesium-calcium-silicate type, for example wollastonite. Talc is a particularly preferred filler. The fillers are preferably coated, preferably with stearic acid or stearates. This positively influences the trickling behavior.

The fillers are preferably used in an amount of from 0 to 60 wt. %, in particular from 0 to 50 wt. %, preferably 0.1 to 40 wt. %, particularly preferably 1 to 30 wt. %, in each case based on the mass of the entire thermally expandable composition.

Chromophoric components, in particular black dyes based on graphite and/or carbon black, are contained in the thermally expandable compositions according to the invention preferably in an amount of from 0 to 2 wt. %, in particular from 0.1 to 0.8 wt. %, very particularly preferably 0.15 to 0.4 wt. %, in each case based on the mass of the entire thermally expandable composition.

It is possible to use, as antioxidants or stabilizers, for example, sterically hindered phenols and/or sterically hindered thioethers and/or sterically hindered aromatic amines, for example bis-(3,3-bis-(4′-hydroxy-3-tert-butylphenyl) butanoic acid) glycol ester or also 4-methylphenol, reaction product with dicyclopentadiene and isobutylene (Wingstay L).

Antioxidants or stabilizers are preferably contained in the thermally expandable compositions according to the invention in an amount of from 0 to 0.5 wt. %, in particular from 0.1 to 0.3 wt. %, in each case based on the mass of the entire thermally expandable composition.

Reactive diluents for epoxy resins are epoxy group-containing, low-viscosity substances (glycidyl ethers or glycidyl esters) having an aliphatic or aromatic structure. These reactive diluents can be used to lower the viscosity of the binder system above the softening point, and can control the pre-gelation process during injection molding. Typical examples of suitable reactive diluents are mono-, di- or triglycidyl ethers of C6 to C14 monoalcohols or alkyl phenols and the monoglycidyl ethers of cashew nut shell oil, diglycidyl ethers of ethylene glycol, diethylene glycol, triethylene glycol, tetraethylene glycol, propylene glycol, dipropylene glycol, tripropylene glycol, tetrapropylene glycol, 1,4-butylene glycol, 1,5-pentanediol, 1,6-hexanediol, cyclohexane dimethanol, triglycidyl ethers of trimethylolpropane and the glycidyl esters of C6 to C24 carboxylic acids or mixtures thereof.

The thermally expandable compositions according to the invention are preferably formulated such that they are solid at 22° C. A thermally expandable composition is referred to as “solid” according to the invention if the geometry of this composition does not deform under the influence of gravity at the indicated temperature within 1 hour, in particular within 24 hours.

The thermally expandable compositions according to the invention can be prepared by mixing the selected components in any suitable mixer, such as a kneader, a double-Z kneader, an internal mixer, a twin-screw mixer, a continuous mixer, or an extruder, in particular a twin-screw extruder.

Although it may be advantageous to slightly heat the components to facilitate achieving a homogeneous and uniform compound, care has to be taken to ensure that temperatures which cause activation of the curing or foaming are not reached. The resulting thermally expandable composition can be shaped immediately after its preparation, for example by blow molding, pelletizing, injection molding, compression molding, stamping or extrusion.

The thermally expandable composition is expanded by heating, the composition being heated for a specific time to a specific temperature sufficient to cause the activation of the carbonate. The expansion takes place while releasing CO₂ from the cyclic carbonates. Depending on the composition of the expandable formulation and the conditions of the production line, temperatures of this kind are usually in the range of 110 to 240° C., preferably 120 to 210° C., with a dwell time of from 10 to 90 minutes, preferably from 5 to 60 minutes.

In the field of vehicle construction, it is particularly advantageous for the compositions according to the invention to expand when the vehicle passes through the furnace for hardening the cathodic dip coating, and therefore a separate heating step can be omitted.

The thermally expandable compositions of the present invention can be used in a wide range of support, filling, sealing, and adhesive applications, for example in the field of baffle parts for sealing hollow spaces in vehicles or as molded parts that stiffen structures. However, it is also conceivable to use said compositions as an underlay adhesive, for example in the door or roof region. For an intended use of this kind, the thermally expandable compositions according to the invention can be applied by means of direct extrusion. However, the compositions can also be brought into extruded form on the application site, pressed thereon by heating the steel, and melted. As a third alternative, application as a co-extrudate is also conceivable. In this embodiment, according to the invention, a second adhesive composition is applied in a thin layer under the actual non-adhesive molded part made of the thermally expandable composition. In the context of this embodiment, this second adhesive layer is used to fix the molded part during shell construction.

Accordingly, the thermally expandable compositions are particularly suitable for producing shaped bodies, in particular baffle parts for sealing hollow spaces and/or molded parts that stiffen structures, i.e. for producing parts that are inserted into the hollow spaces of vehicles, then expand by heating and simultaneously cure, and in this way seal and/or strengthen the hollow space as completely as possible.

Accordingly, in a further aspect, the present invention is directed to a shaped body comprising a thermally expandable composition according to the invention. This can be, for example, a baffle part for sealing hollow spaces of a component which has a shape which is adapted to the hollow space, or a structural reinforcement part.

According to the invention, a “shape which is adapted to the hollow space” is in this case understood to mean all geometries of baffle parts that ensure that the hollow space is completely sealed after expansion. In this case, the shape of the baffle part can be individually modeled on the shape of the hollow space and have corresponding tips and/or curves; however, in the case of the thermally expandable compositions according to the invention which have high degrees of expansion, introducing a correspondingly large amount in variable form, for example in the form of a bead or a cut strand of the material, into the hollow space can also be sufficient to ensure that the hollow space is completely sealed after expansion.

Baffle parts of this kind are usually produced from the thermally expandable compositions according to the invention by means of injection molding techniques. The thermally expandable compositions are in this case heated to temperatures in the range of 70 to 120° C. and then injected into a correspondingly shaped mold.

The shaped bodies according to the invention can be used in all products which have hollow spaces. In addition to vehicles, these include aircraft, rail vehicles, domestic appliances, furniture, buildings, walls, partitions or boats, for example.

The present invention also relates to a method for sealing and filling hollow spaces in components, for strengthening or stiffening components, in particular hollow components, and for bonding movable components, using the compositions and shaped bodies described herein. The method is preferably a method for sealing hollow spaces of a component, a baffle part according to the invention being introduced into the hollow space and then being heated to a temperature of greater than 30° C., preferably from 50° C. to 250° C., more preferably 80° C. to 160° C., such that the thermally expandable composition expands and seals, fills or strengthens the hollow space.

The present invention also relates to the use of a shaped body or baffle part according to the invention for acoustically sealing hollow spaces in components and/or for the sealing hollow spaces in components against water and/or moisture.

The present invention also relates to the use of a shaped body according to the invention for strengthening or stiffening components, in particular hollow components.

The following examples are intended to explain the invention in greater detail. The selection of the examples should not limit the scope of the subject matter of the invention. In the compositions, all stated amounts are parts by weight unless indicated otherwise.

### EXAMPLES

Synthesis of an Organic Carbonate from Epoxide and Carbon Dioxide

Tetrabutylammonium iodide was obtained from Acros. Epoxides DER 331 and DER 749 were obtained from Dow Chemicals.

The carbonate was prepared using a two-part 1 liter glass reactor equipped with a hollow mechanical stirrer and a thermometer. The temperature was kept constant by connecting the thermometer to a hot plate. Dry ice (CO₂) was added to another 1 liter round flask. The released gas was introduced into the main reactor through a polyethylene tube via the hollow stirrer. The epoxide was added into the reactor together with the corresponding catalyst (10 wt. % of the epoxide) and heated at 140° C. while stirring constantly. Bubbles could be observed in the epoxy mixture.

The 1 liter round flask was filled with dry ice every 4-5 hours. After 2-3 days, the mixture became white and highly viscous. At this time, the reaction mixture was cooled. It was possible to obtain the carbonates of DER 331 (product of epichlorohydrin with bisphenol A) and DER 749 (product of epichlorohydrin and tetramethylolmethane) as the product.

The synthesis of an organic carbonate starting from polyfunctional epoxides using tetrabutylammonium iodide as a synthesis catalyst is shown by way of example in the following diagram:

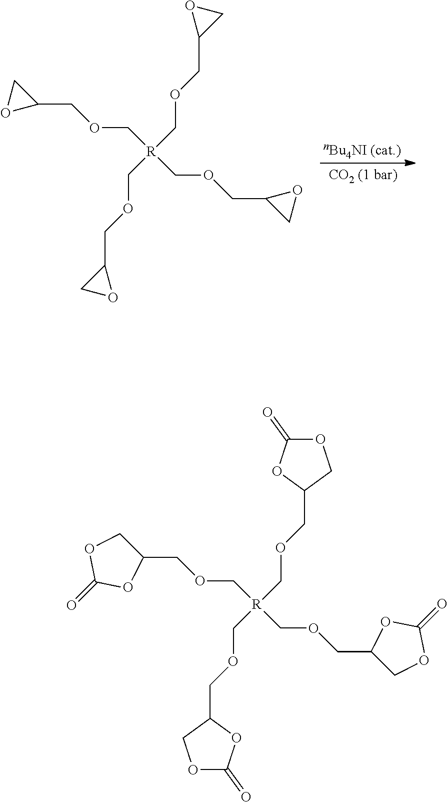

### Expandable Compositions

The expandable compositions were obtained by mixing the constituents listed in the following table (in parts by weight), the base being added last. The expansion and curing was then carried out at 180° C. for 60 min.

| | Example | | | | | | |
|---|---|---|---|---|---|---|---|
| | 1 | 2 | 3 | 4 | 5 | 6 | 7 |
| DER 331 - carbonate | 33.6 | 54.81 | 57 | 57 | | 33 | |
| DER 749 - carbonate | | | | | 14.0 | | 33 |
| DER 331 | 8.4 | | | | 23.6 | 22 | 22 |
| TMPMP | 30 | 30 | 42 | 42 | | | |
| Phenol novolac | | | | | 24.2 | 27.5 | 27.5 |
| Cab-o-Sil TS 720 | 2.5 | 2.5 | | | 2.0 | 2.5 | 2.5 |
| (pyrogenic silicic acid) | | | | | | | |
| Barium sulfate | 24.5 | 11.69 | | | 15.0 | 11.7 | 11.7 |
| Monarch 580 (carbon | | | | | 1.0 | | |
| Glass fibers (~2 mm long) | | | | | 10.0 | | |
| Glass hollow spheres | | | | | 7.0 | | |
| 2-ethyl-4-methylimidazole | 1 | 1 | | | | | |
| Sodium hydride (35% on | | | 1 | | | | |
| mineral oil) | | | | | | | |
| Sodium hydroxide | | | | 1 | | | |
| (finely ground) | | | | | | | |
| 1,1-dimethyl urea | | | | | 3.2 | 3.3 | 3.3 |

TMPMP: Trimethylolpropane tris(3-mercaptopropionate)

The resulting foams are characterized by a high, achievable expansion rate. Furthermore, the foams based on cyclic organic carbonates are characterized by very clean decomposition. It was not possible to detect any decomposition products from the carbonate.

1. A thermally expandable composition comprising
(1) at least one organic compound comprising at least two cyclic carbonate groups,
(2) at least one compound having at least one functional group having a pKa value of greater than 3, and
(3) at least one base.

2. The thermally expandable composition according to claim 1, wherein the organic compound is a polyether and/or polyester, to which at least two cyclic carbonate groups are bonded.

3. The thermally expandable composition according to claim 1, wherein the at least one organic compound is a cyclic organic carbonate of Formula (I) 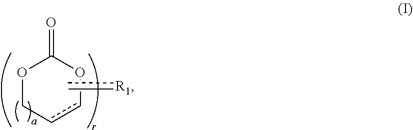 [symbol]
being a single bond or a double bond, where if the ring contains a double bond, R₁ is not bonded by an exo double bond, but rather by a single bond, and vice versa, and R₁ is a linear or branched, substituted or unsubstituted alkyl, linear or branched, substituted or unsubstituted heteroalkyl, linear or branched, substituted or unsubstituted alkenyl, linear or branched, substituted or unsubstituted alkinyl, substituted or unsubstituted cycloalkyl, substituted or unsubstituted aryl, or —C(O)—R^{a}, where R^{a} is H, a linear or branched, substituted or unsubstituted alkyl, linear or branched, substituted or unsubstituted heteroalkyl, linear or branched, substituted or unsubstituted alkenyl, linear or branched, substituted or unsubstituted alkinyl, substituted or unsubstituted cycloalkyl, substituted or unsubstituted aryl, a is an integer from 0 to 5, and r is a natural number from 2 to 10.

4. The thermally expandable composition according to claim 3, wherein the at least one organic compound is a cyclic organic carbonate of Formula (II) 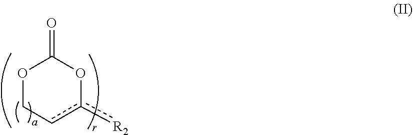 [symbol]
, a and r being as defined above and R₂ being defined as R₁.

5. The thermally expandable composition according to claim 4, wherein the at least one organic compound is a cyclic organic carbonate of Formula (III) or (IV), 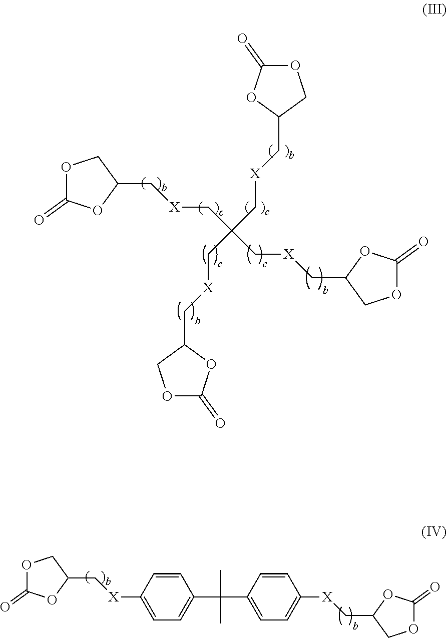
each b and c being, independently of one another, a natural number from 1 to 5, and each X being independently selected from the group consisting of O, S and N.

6. The thermally expandable composition according to claim 5, wherein the at least one organic compound is a cyclic organic carbonate of Formula (V) or (VI)

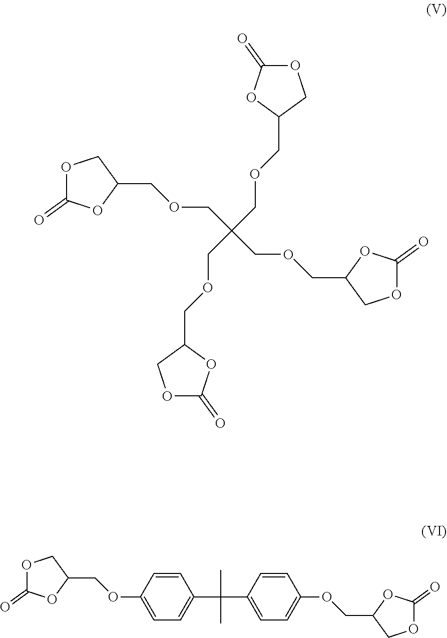 [SMILES: O=C1OCC(COCC(COCC2COC(=O)O2)(COCC2COC(=O)O2)COCC2COC(=O)O2)O1 ; CC(C)(c1ccc(OCC2COC(=O)O2)cc1)c1ccc(OCC2COC(=O)O2)cc1]

7. The thermally expandable composition according to claim 1, wherein the at least one base is a nitrogen-containing base.

8. The thermally expandable composition according to claim 7, wherein the at least one base is selected from the group consisting of 1-methylimidazole, 2,4-ethylmethylimidazole (EMI), 1,4-diazabicyclo[2.2.2]octane (DABCO), 1,8-diazabicyclo[5.4.0]undec-7-ene (DBU), 1,5-diazabicyclo[3.4.0]non-5-ene (DBN) and mixtures thereof.

9. The thermally expandable composition according to claim 1, wherein the thermally expandable composition comprises, based on the binder system (total weight of the composition prior to expansion without fillers), 20 to 90 wt. % of the at least one organic compound comprising at least two cyclic carbonate groups.

10. The thermally expandable composition according to claim 1, wherein the at least one functional group of the at least one compound is selected from OH, NH₂ and/or SH groups.

11. The thermally expandable composition according to claim 1, wherein the at least one compound has at least two functional groups selected from OH and/or SH groups, having a pKa value of greater than 5.

12. A shaped body, wherein the shaped body comprises a thermally expandable composition according to claim 1.

13. A method for sealing and filling hollow spaces in components, for strengthening or stiffening components, and/or for bonding movable components, using a thermally expandable composition according to claim 1.

14. A method for sealing and filling hollow spaces in components and for strengthening or stiffening components, comprising steps of: inserting a shaped body according to claim 12 into a hollow space of a component; and heating the shaped body to a temperature of greater than 30° C., such that the thermally expandable composition expands and seals, fills, strengthens or stiffens the component.

15. A method for sealing and filling hollow spaces in components, for strengthening or stiffening components, and/or for bonding movable components, contacting the component(s) to be sealed, filled, strengthened, stiffened and/or bonded with the shaped body according to claim 12 and heating the shaped body to a temperature sufficient to activate the thermally expandable composition such that the thermally expandable composition expands and seals, fills, strengthens, stiffens and/or bonds the component(s).

16. The use of a shaped body according to claim 12 for acoustically sealing hollow spaces in components and/or for sealing hollow spaces in components against water and/or moisture, or for strengthening or stiffening components.
